(19) United States
(12) Patent Application Publication
(10) Pub. No.: US 2005/0234525 A1
(43) Pub. Date: Oct. 20, 2005

(54) Electric stimulation for treating neuropathy using asymmetric biphasic signals

(76) Inventor: David B. Phillips, Charles Town, WV (US)

Correspondence Address: HOWREY LLP, C/O IP DOCKETING DEPARTMENT, 2941 FAIRVIEW PARK DR, SUITE 200, FALLS CHURCH, VA, 22042-2924, US

(21) Appl. No.: 10/823,774

(22) Filed: Apr. 14, 2004

(51) Int. Cl. A61N 1/00; A61N 1/08; A61N 1/10; A61N 1/18; A61N 1/20

(52) U.S. Cl. 607/68; 607/2

(57) ABSTRACT

The inventions include methods for treating neuropathy and apparatus for use in the methods. The apparatus may include a controller configured to output an asymmetric biphasic signal. The apparatus can also include a first container and a second container. The first and the second containers can be configured to hold a fluid. The apparatus may also include a first electrode and a second electrode. The first electrode and the second electrode can be configured to be in electrical contract with the fluid held by the container, and can be configured to be coupled to the controller. The electrodes may be configured to receive the asymmetric biphasic signal output from the controller.

### FIELD OF THE INVENTION

The invention relates generally to devices and methods for electro-stimulation of nerves and muscles and for the treatment of neuropathy. More particularly, the invention relates to the treatment of neuropathy using by electro-stimulation of muscles and nerves using an asymmetric biphasic signal.

### BACKGROUND OF THE INVENTION

There are various causes of pain in the feet and lower legs. Whatever the cause, the effects may, in some instances, be unbearable or even immobilizing. Once cause of pain in the extremities is peripheral neuropathy, a disorder of the peripheral nerves, which connect the central nervous system (CNS) to the nerves throughout the rest of the body. Peripheral neuropathy can cause progressive nerve damage that may result, in some cases, in impaired functioning of certain internal organs. Symptoms may begin with slight tingling, numbness, or pain in the hands, feet, or legs. These symptoms can culminate in severe pain and weakness of the muscles. Eventually, many individuals suffering from peripheral neuropathy completely loose sensation in the affected areas. One of the principal causes of peripheral neuropathy is diabetes. Peripheral neuropathy can, however, result from a number of different disorders including poor circulation, cardiovascular disease, trauma to the lumbar area, and immunodiffenciency diseases. Additionally, the administration of certain drugs can lead to peripheral neuropathy, including, for example, chemotherapy drugs, hypertension drugs, and drugs for the treatment of AIDS.

Treatment of neuropathy has largely centered on removing the cause of the neuropathy and taking special care of affected extremities. But for many patients, the causes of neuropathy are the very drugs that they rely upon to treat life-threatening illnesses. Other patients' neuropathies are too advanced to reverse their effects, even after the underlying cause of the neuropathy is removed. Still other patients find that the most common drug treatments prescribed for pain relief—analgesics, anticonvulsants, and low-dose antidepressants—do not provide adequate pain relief.

The prior art to date has found no treatment that can reverse the effects of advanced peripheral neuropathy. While electrical stimulation devices have been used in a variety of medical contexts for stimulating muscles and for relieving pain, these devices have not generally been successful in substantially relieving and treating peripheral neuropathy.

Electric muscle stimulation (EMS) devices apply a voltage across a muscle and cause the muscle to contract. By applying pulses of electric current, EMS devices can cause successive cycles of contraction and relaxation. EMS devices are commonly used in physical therapy and rehabilitation for muscle reeducation and training, and to prevent muscle atrophy during periods of convalescence. Other uses of EMS therapy include muscle spasm reduction, circulation improvement, and treatment of scoliosis and incontinence. EMS devices typically require placing localized electrodes across a muscle, with one of the electrodes being placed as close as possible to the muscle's motor point in order to facilitate strong, clean muscle contractions.

Traditional transcutaneous electrical neural stimulation (TENS) devices work in a similar manner as EMS devices in that a voltage is applied across electrodes applied to an affected area. TENS devices typically apply a smaller voltage for a longer duration, than do EMS devices, and typically apply more current than EMS devices. The difference in operation is a result of the different objectives of the two types of devices. While EMS devices are designed to stimulate muscle contraction, TENS devices are designed to stimulate nerves without necessarily stimulating and accompanying muscle contraction.

The complex phenomenon of pain is not well understood, but one prevailing theory about the mechanism for feeling pain is the gate control theory. Under this theory, chronic pain is the result of uncontrolled signaling of nerve fibers called C-fibers. TENS devices are thought to bring relief from chronic pain by stimulation of a different class of nerve fibers called A-fibers. The stimulation of the A-fibers is believed to prevent the C-fibers from signaling by “closing the gate” or resetting the C-fibers to an inactive state. While EMS devices have been used to alleviate neuropathic pain, the relief has been found by many patients to be inadequate, and such devices have never successfully reversed advanced neuropathy.

Patients experiencing chronic pain from peripheral neuropathy thus have a pressing need for a device that can provide substantial relief from their pain, and methods that can treat and, in many cases, reverse the effects of advanced neuropathy.

### SUMMARY OF THE INVENTION

In light of the above-identified deficiencies of contemporary systems, it is thus an object of the present invention to provide a system and method to addresses some of the problems associated with treating, and in some cases, reversing damage and pain caused by peripheral neuropathy.

In a first aspect of the invention, an apparatus that may be used to treat neuropathy may include a controller. The controller may be configured to output an asymmetric biphasic signal. The apparatus can also include a first container and a second container. The first and the second containers can be configured to hold a fluid. The apparatus may also include a first electrode and a second electrode. The first electrode and the second electrode can be configured to be in electrical contract with the fluid held by the container, and can be configured to be coupled to the controller. The electrodes may be configured to receive the asymmetric biphasic signal output from the controller.

In one embodiment, the asymmetric biphasic signal may be generated using a transformer. In another embodiment, the fluid may be water. In another embodiment of the invention, the fluid may be a water-electrolyte solution. In yet another embodiment, the fluid may be a water-electrolyte solution including at least one of potassium, calcium, benfotiamine, magnesium, and colloidal silver.

In an alternative embodiment, the controller may be configured to selectively output the asymmetrical biphasic signal in a number of modes, including a pulsed mode, a continuous wave mode, and a surged mode. In yet another embodiment, the first container and the second container can be part of a unitary structure. Alternatively, the first container and the second container may be separate containers. In yet another embodiment, the asymmetric biphasic signal may be output at approximately 7.83 Hz.

In a second aspect of the invention, a method can include outputting an asymmetric biphasic signal. The asymmetric biphasic signal may be received at a first electrode and a second electrode. The first electrode and the second electrode can be configured to be in electrical contact with a fluid disposed within a first container and a second container, respectively.

In one embodiment, the method may include selectively outputting the asymmetric biphasic signal in one of a pulsed mode, a continuous wave mode, and a surged mode. In another embodiment, the method may include adding electrolytes to water disposed within the first container and the second container. In yet another embodiment, the method may include adding at least one of magnesium, calcium, potassium, and benfotiamine to the water disposed in the first container and the second container. In another embodiment of the invention, the method may include propagating the asymmetric biphasic signal through a first extremity and a second extremity. In yet another embodiment, the asymmetric biphasic signal may be output at a frequency of approximately 7.83 Hz.

In a third aspect of the invention, a method of treating neuropathy may include placing a first extremity in a first fluid-filled container and placing a second extremity in a second fluid-filled container. An asymmetric biphasic signal can be output from a controller to the first extremity and the second extremity via the fluid in the first container and the fluid in the second container.

In one embodiment of the invention, the method of treating neuropathy may include removing the first extremity from the container and applying a topical cream to the first extremity. In another embodiment, the method of treating neuropathy may include applying a topical cream including menthol and camphor to the first extremity. In yet another embodiment, a method of treating neuropathy may include outputting an asymmetric biphasic signal selectively in one of a pulsed mode, a continuous wave mode, and a surged mode. In another embodiment, the method of treating neuropathy may include adding electrolytes to the fluid in the first container. In another embodiment, the method of treating neuropathy may include adding at least one of magnesium, calcium, potassium, and benfotiamine to the fluid in the first container.

### BRIEF DESCRIPTION OF THE DRAWINGS

While the specification concludes with claims particularly pointing out and distinctly claiming the invention, it is believed the same will be better understood from the following description taken in conjunction with the accompanying drawings, which illustrate, in a non-limiting fashion, the best mode presently contemplated for carrying out the present invention, and in which like reference numerals designate like parts throughout the Figures wherein:

FIG. 1 shows a functional block diagram illustrating an apparatus for treating neuropathy according to one embodiment of the invention;

FIG. 2 shows a graph illustrating a surged mode output signal according to an embodiment of the invention;

FIG. 3 shows a graph illustrating a continuous wave output signal according to one embodiment of the invention;

FIG. 4 shows a graph illustrating a pulsed wave output signal according to one embodiment of the invention;

FIG. 5 shows a perspective view of a controller according to an embodiment of the invention;

FIG. 6 shows an exemplary circuit board layout for a controller according to an embodiment of the invention;

FIG. 7 shows an partial cross-sectional view of an apparatus for treating neuropathy according to one embodiment of the invention;

FIG. 8 shows a sock that may be used to treat neuropathy according to an embodiment of the invention;

FIG. 9 shows a glove that may be used to treat neuropathy according to another embodiment of the invention; and

FIG. 10 shows a flowchart of a method of treating neuropathy according to an embodiment of the invention.

### DETAILED DESCRIPTION

The present disclosure will now be described more fully with reference to the Figures in which embodiments of the present invention are shown. The subject matter of this disclosure may, however, be embodied in many different forms and should not be construed as being limited to the embodiments set forth herein.

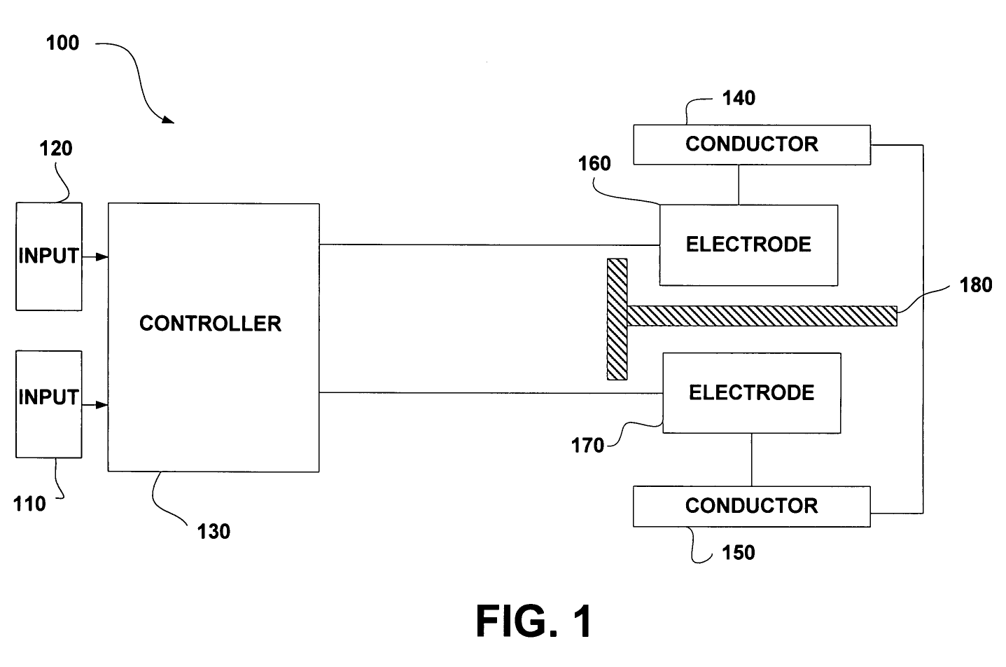

FIG. 1 is a functional block diagram illustrating an apparatus for treating neuropathy according to one embodiment of the invention. The apparatus for treating neuropathy 100 may include a first input 110 and a second input 120. The first input 110 and the second input 120 may be electrically coupled to controller 130. Controller may be electrically coupled to electrode 160 and electrode 170. Electrode 160 may be coupled to conductor 140. Likewise, electrode 170 can be coupled to conductor 150. Electrode 160 and electrode 170 may be isolated from one another by a dielectric or insulating material 180, such that signals may pass between electrode 160 and electrode 170 via conductor 140 and conductor 150.

A user can use first input 110 and second input 120 to provide instructions to controller 130. In one embodiment, input 110 can be used to control the intensity of the signal output from controller 130, and input 120 can be used to change a controller mode. For example, controller 130 may be configured to output a signal in one of three modes: a pulsed mode, a surged mode, and a continuous wave mode, based on the input received from, for example, input 120.

Input 110 and input 120 may be, for example, rotary knobs, switches, dials, buttons, levers, or other known input devices. Input 110 and input 120 may also include, for example, an analog or digital input for receiving instructions from, for example, a processor coupled to the controller. Any type of input may be used to provide instructions to the controller.

Controller 130 can be configured to output an asymmetrical biphasic signal. In one embodiment, the controller may be configured to output the asymmetrical biphasic signal in multiple operational modes, such as, for example, a pulsed mode, a continuous wave mode, and a surged mode. In one embodiment, these modes can correspond to an operational mode in which the asymmetrical biphasic signal is surged at a frequency of approximately 7.83 Hz, an EMS mode, and a TENS mode, respectively, as will be described in further detail below

In another embodiment, controller 130 may be configured to output the asymmetrical biphasic signal using a number of predetermined programs. In one embodiment, a predetermined program can include, for example, outputting an EMS signal and a TENS signal in a predetermined sequence. In an embodiment of the invention, an EMS signal may be output for a duration of, for example, 4 seconds, and the TENS signal may be output for a duration of, for example, 1 second. In an alternative embodiment, the EMS signal may be output for a duration of, for example, 1 second and the TENS signal may be output for a duration of, for example, 4 seconds. In yet another embodiment, the EMS signal may be output for a duration of, for example, 4 seconds, and the TENS signal may be output for a duration of, for example, 1 second. This pattern may continue for, for example, three or four cycles, followed by the EMS signal being output for a duration of, for example, 1 second, followed by the TENS signal being output for a duration of, for example, 4 seconds. Any number of cycles can be included in a predetermined program.

Controller 130 may be configured to include a memory device (not illustrated) in which the predetermined programs may be stored. Multiple predetermined programs may be stored in the memory device. The memory device may be configured to receive predetermined programs from, for example, a host computer via a bus. The host computer may be configured to download a predetermined program to a memory device located within a controller. In yet another embodiment, the controller may include a removable memory device, such as, for example, a flash memory device. The use of a rewritable memory device may allow for predetermined programs to be customized for a particular user.

In an alternative embodiment, controller 130 need not include a memory device, and may instead include hard-wired programs. In yet another embodiment, controller need not include any predetermined programs and may be configured to output in three different modes, such as, for example, an EMS mode, a TENS mode, and an approximately 7.83 Hz mode.

Controller 130 outputs an asymmetric biphasic signal to a first electrode 160 and a second electrode 170. The electrodes may be in electrical contact with controller 130. Electrodes can be made of any conductive material. In one embodiment, electrode 160 and electrode 170 may be removably coupled to the controller such that they may be easily replaced. In some embodiments of the invention, first electrode 160 and second electrode 170 can be corrosion-resistant.

First electrode may be electrically coupled to first conductor 140. Second electrode 170 can be electrically coupled to second conductor 150. The first conductor 140 and the second conductor 150 can be electrically coupled to one another. Electrode 160 and electrode 170 can be electrically isolated from one another by, for example, an insulator 180, such that the asymmetric biphasic signal can propagate between electrode 160 and electrode 170 via conductor 140 and conductor 150.

In one embodiment of the invention, conductor 140 and conductor 150 may be, for example, extremities of a body. In some embodiments, the conductor 140 and conductor 150 can be, for example, feet and legs. In an alternative embodiment, conductor 140 and conductor 150 can be, for example, hands. By placing the electrodes in electrical contact with the extremities, a circuit may be completed through which the asymmetric biphasic signal output from controller 130 may be propagated.

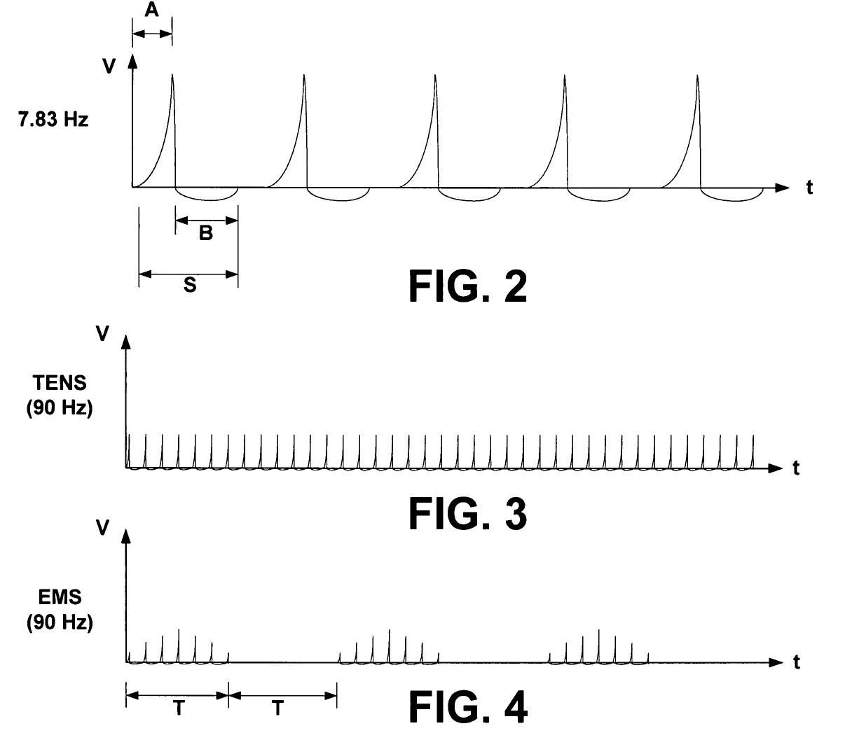

FIG. 2 is a graph illustrating a surged mode output according to an embodiment of the invention. In one embodiment, controller 130 can be configured to output an asymmetric biphasic signal “S.” The asymmetric biphasic signal “S” has a waveform including a thin positive portion “A” and a wide negative portion “B.” Thin positive portion “A” has a relatively short duration and can be used to stimulate the nerves. In one embodiment, the thin positive portion “A” of the asymmetric biphasic signal “S” may have, for example, a relatively low current under the curve and may have a relatively high transient voltage, as illustrated in FIG. 2. In one embodiment, the transient voltage can be, for example, between 40 and 90 volts. In another embodiment, the voltage can be between approximately 0 and approximately 90 VAC. A number of different voltage ranges may be used, as long as the voltage is tolerable and the duration of the thin portion of the asymmetric biphasic signal “A” is not so large as to stimulate muscle contractions. In yet another embodiment, the transient voltage may be controllable by a user, using for example, input 110. In yet another embodiment, the current under the curve can be much less than what is used in traditional TENS systems.

The wider portion “B” of the asymmetric biphasic signal “S” can be temporally longer than the thin portion “A” of the asymmetric biphasic signal “S” and can be used, for example, to stimulate the muscle cells. In one embodiment, the wide portion of the asymmetric biphasic signal may have a greater amount of current under the curve than the thin portion “A” and may have a lower transient voltage than the thin portion “A.” The transient voltage may be, for example, between 5 and 20 VAC. In another embodiment of the invention, the transient voltage of the wide portion “B” of the asymmetric biphasic signal may have a transient voltage between 0 and 40 VAC. In yet another embodiment, the asymmetric biphasic signal can have a transient voltage that may controllable by, for example, a user, using, for example, input 110.

In the embodiment illustrated in FIG. 2, the asymmetric biphasic signal “S” may be output at a frequency of approximately 7.83 Hz. In yet another embodiment, the frequency of the asymmetrical biphasic signal may be predetermined and preprogrammed or hardwired into the controller. Any frequency may be acceptable for outputting the asymmetrical biphasic signal. As described above, the asymmetric biphasic signal may be configured to stimulate both muscle tissue and nerve tiussue to thereby treat neuropathy. The asymmetric biphasic signal may also exhibit an additional DC component, due in some cases to the natural operation of the transistor used in connection with some embodiments of the invention to generate the asymmetric biphasic signal. The DC component may be configured to alter the resting/action potentials of the nerve cells, which may, in some instances, produce therapeutic effects.

FIG. 3 is a graph illustrating a continuous wave output according to one embodiment of the invention. In this embodiment, the asymmetric biphasic waveform may be output in a continuous wave mode. In one embodiment, the asymmetrical biphasic signal can be output at approximately 90 Hz. Alternatively, the asymmetric biphasic signal can be output at approximately 100 Hz. The asymmetric biphasic signal can be output at any acceptable frequency.

FIG. 4 is a graph illustrating a pulsed wave output according to an embodiment of the invention. As illustrated in FIG. 4, asymmetric biphasic signal can be output such that a pulse train is output every five seconds. The pulse train can have a duration of “T.” In one embodiment, the pulse train may have a period of 5 seconds. Any pulse train duration may be used. In one embodiment, the asymmetrical biphasic signal can have a voltage that varies from pulse to pulse for the duration of the pulse train. A illustrated in FIG. 2, in some embodiments, the center pulse in the pulse train can have the peak voltage with prior pulses having ascending transient voltages, and subsequent pulses having descending transient voltages.

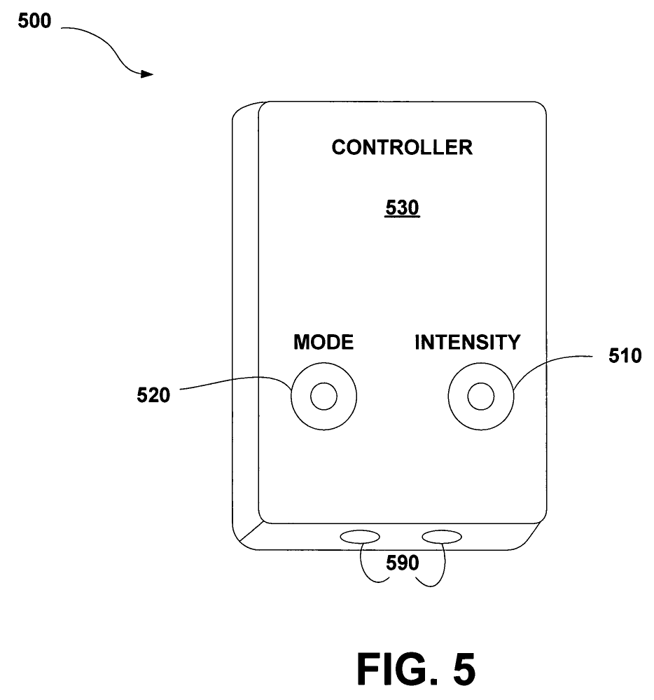

FIG. 5 is a perspective view of a control unit 500 according to an embodiment of the invention. Controller 530 may include a first knob 510 and a second knob 520. Controller 530 may also have at least one output port 590.

First knob 5 10 may be, for example, an intensity control. First knob 510 may be configured to permit a user to control the intensity of the asymmetrical biphasic signal, by, for example, controlling the voltage of the asymmetrical biphasic signal output by the controller 530. Controller 530 can also include a second knob 520. Second knob 520 can be configured to change the output mode of the controller. In one embodiment, the second knob 520 can be configured such that the controller can output an asymmetrical biphasic signal in either a continuous wave mode, a pulsed wave mode, or a surged mode.

Control unit 500 may include a power source, such as, for example, a 9V battery. In an alternative embodiment, control unit 500 may include a power cable configured to be plugged into, for example, a wall outlet or other AC power source. Control unit 500 can be configured to receive power from any acceptable power source.

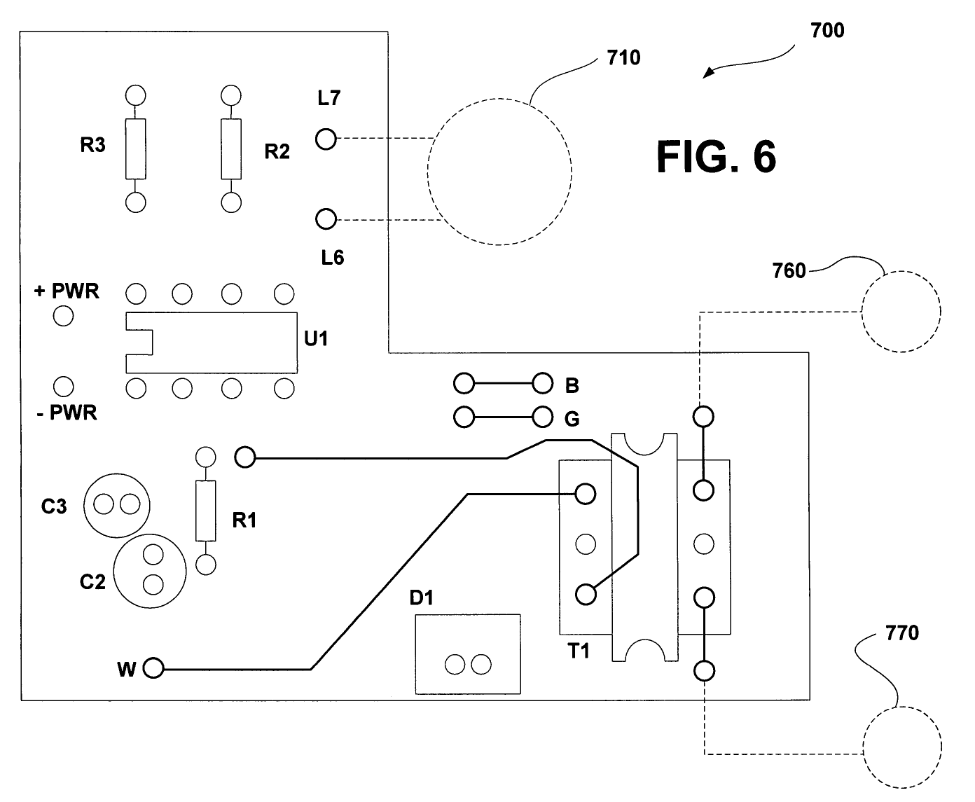

FIG. 6 shows an exemplary circuit board layout for a controller according to an embodiment of the invention. The control circuit 700 can be housed within the controller. In one embodiment, controller can include a timer, “U1.” In one embodiment, timer can include, for example, a 555 timer. Any type of timer may be used in accordance with the invention. Control circuit 700 may include a potentiometer 710. Potentiometer 710 may be used to adjust the voltage differential across terminals L6 and L7, and may thereby control the intensity of the asymmetrical biphasic signal output from the circuit. Potentiometer 710 is illustrated in broken lines to connote the fact that it does not need to be physically located on the circuit board assembly 700.

Control circuit 700 can also include a transistor “T1.” In one embodiment, transistor TI may be used to generate the asymmetrical biphasic signal output from the controller. Transformer TI can be, for example, an audio transformer. Audio transformers often suffer from a deleterious effect known as ringing. One embodiment of the present invention may utilize use this ringing effect and may capitalize on the effect to generate the asymmetrical biphasic signal. In another embodiment, the asymmetric biphasic signal can be generated using digital signal generators, or other analog or digital signal generating means.

In one embodiment, the patient is electrically isolated from the circuitry, including the power source using, for example, the transformer. In an alternative embodiment, the patient is electrically isolated from the circuitry, including the power source using, for example, digital circuitry. In yet another embodiment of the invention, the patient may be electrically isolated from the circuitry using, for example, a fiber optic system.

The transformer T1 can be electrically coupled to electrodes 760 and 770. Thus, the asymmetric biphasic signal output from the control circuit 700 can be output from electrode 760 and electrode 770 to, for example, a patient for the treatment of neuropathy. In one embodiment of the invention, the device may effectively reads the effective impedance of, for example, the patient, and may be configured to automatically adjust the amount of voltage supplied by the power source based on the impedance or resistivity of the patient. This may be performed using either digital or analog circuitry. In one embodiment, a circuit as illustrated in FIG. 6 may be used to perform this function. Numerous other analog or digital circuits may be utilized to perform this function.

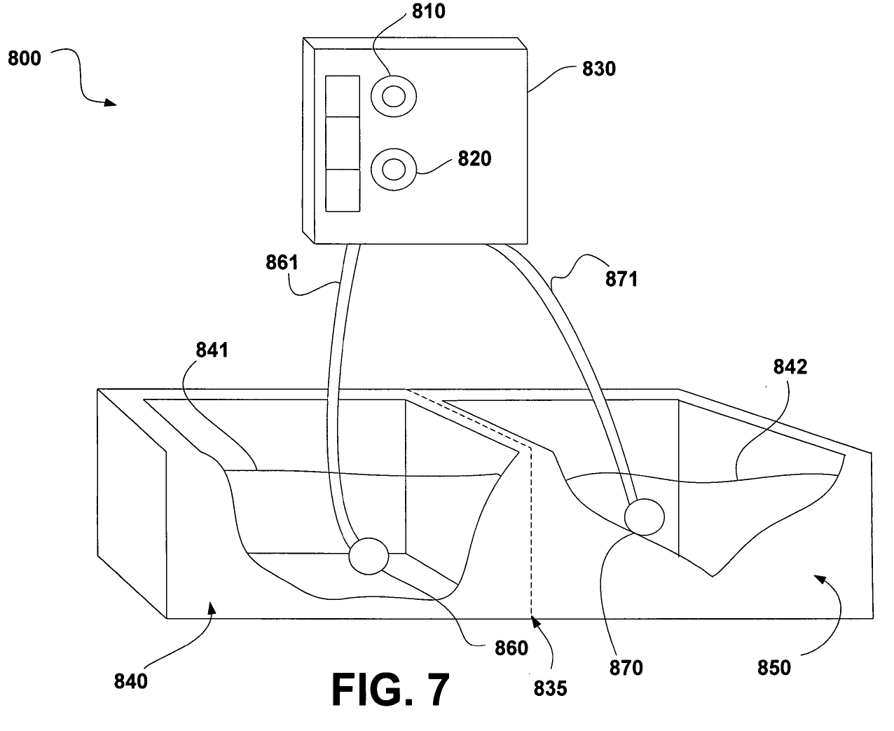

FIG. 7 is a partial cross-sectional view of an apparatus for treating neuropathy 800 according to one embodiment of the invention. An apparatus for treating neuropathy 800 can include a controller 830. Controller 830 may include a first input 810 and a second input 820. The first input 810 and the second input 820 may be, for example, rotary knobs or dials. Alternatively, first input 810 and second input 820 may be switches, buttons or any input means. As discussed above, controller 830 can be configured to output an asymmetrical biphasic signal.

Controller 830 may be coupled to a first electrode 860 and a second electrode 870 such that the first electrode 860 and the second electrode 870 receive the asymmetrical biphasic signal output by controller 830. Controller 830 can be coupled to the first electrode 860 and the second electrode, by, for example, first lead 861 and second lead 871, respectively. In one embodiment, the first lead 861 and the second lead 871 are removably coupled to the controller 830. In another embodiment, first lead 861 and second lead 871 may be affixed to controller 830.

First electrode 860 and second electrode 870 may be in electrical contact with a first fluid 841 and a second fluid 842. The first fluid 841 and the second fluid 842 can be housed in, for example, a first container 840 and a second container 840, respectively. In an alternative embodiment, the fluid can be substantially contained in a fluid-absorbing medium, such as for example, a sponge, which can be configured to contact a patient. The first container can be separated from the second container by an insulator, which is indicated in FIG. 7 by a dashed line 835. In one embodiment, the first container 840 and the second container 850 are formed from a unitary structure. In an alternative embodiment, the first container 840 and the second container 850 are formed from separate structures.

In one embodiment, first fluid 841 and second fluid may be water. In another embodiment, the first fluid 841 and the second fluid may be a water-electrolyte solution. In yet another embodiment, first fluid 841 and second fluid are the same. In an alternative embodiment, first fluid 841 and second fluid 842 may be different types of fluid.

In one embodiment, a water-electrolyte solution can include, for example, colloidal silver, potassium, calcium, benfotiamine, and Epsom salts, which are a source of magnesium. In an alternative embodiment, a water-electrolyte solution may include at least one of colloidal silver, potassium, calcium, benfotiamine, or magnesium. In yet another embodiment, the fluid may include tea tree oil. In one embodiment, an 8 ounce container of electrolytes and minerals for adding to the fluid can include colloidal silver at a concentration of 20 ppm; 90 mg of potassium, 100 mg of calcium, 1000 mg of benfotiamine, and 450 mg of Epsom salts. In one embodiment, this exemplary electrolyte solution may have a tolerance of ±30%. Water-electrolyte solution may include any number of different types of ions and may include, for example, only one ion. One acceptable electrolyte mixture for use with the present invention can be purchased from Rebuilder Medical, Inc., Charles Town, West Va.

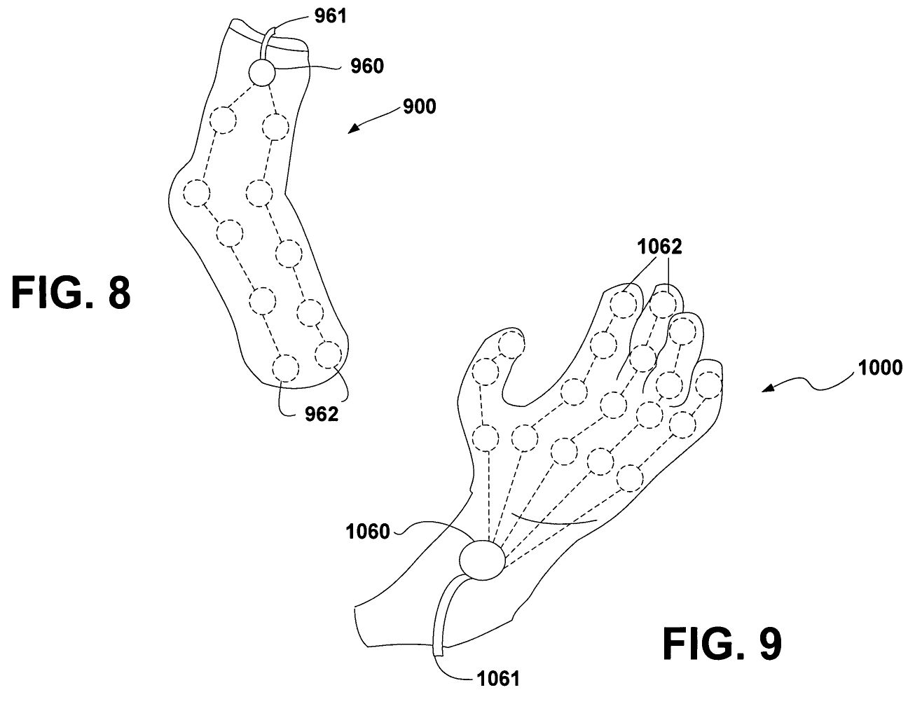

FIG. 8 shows a sock 900 that may be used to treat neuropathy according to another embodiment of the invention. A sock 900 used for treating neuropathy may include an electrode 960 configured to receive an asymmetric biphasic signal from a controller (not illustrated). In one embodiment, the electrode 960 can be coupled to a number of other electrodes 962 within the sock 900. In one embodiment, electrode 960 can be coupled to the controller (not shown) via a lead 961.

In an alternative embodiment, a single electrode 960 can be coupled to the sock 900 and sock 900 can be sprayed with a water-electrolyte solution such that the sock 900 may transmit the asymmetric biphasic signal from the controller to the foot within the sock. In yet another embodiment, the sock 900 can be sprayed with a water-electrolyte solution and can include a number of electrodes 962 within the sock 900.

FIG. 9 shows a glove 1000 that may be used to treat neuropathy according to another embodiment of the invention. Glove 1000 may operate in a similar manner as sock 900, described above. Glove 1000 may include an electrode 1060 that may be coupled to the controller (not illustrated). In one embodiment, glove 1000 may be removably coupled to the controller via a lead 1061. In one embodiment, glove 1000 can include a number of electrodes 1062 within the glove 1000.

In an alternative embodiment, a single electrode 1060 may be coupled to the glove 1000 and the glove 1000 can be sprayed with a water-electrolyte solution such that the glove 1000 may transmit the asymmetric biphasic signal from the controller to the hand within the glove. In yet another embodiment, the glove 1000 may be sprayed with the water-electrolyte solution and can include a number of electrodes 1062 within the glove 1000.

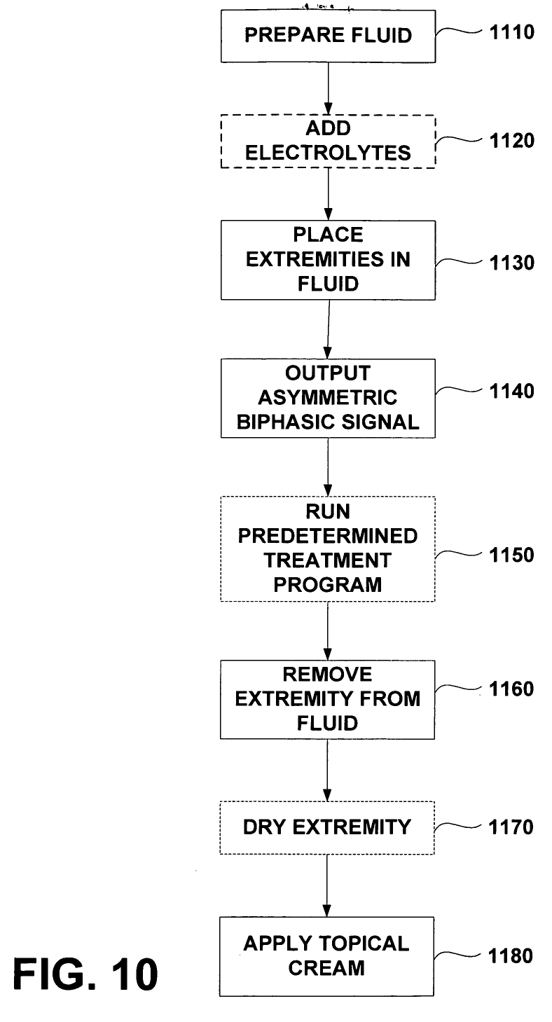

FIG. 10 is a flowchart of a method of treating neuropathy according to an embodiment of the invention. A method of treating neuropathy according to one embodiment of the invention may include preparing the fluid to be placed into the first and second containers, step 1110. Preparing the fluid, step 1110, may include filling the first container and the second container with warm water. In another embodiment of the invention, electrolytes may be added to the warm water, as illustrated in optional step 1120. After the fluid has been prepared, step 1110 and, in some embodiments, the electrolytes have been added to the fluid, 1120, the extremities to be treated may be placed in the fluid, step 1130. After the extremities have been placed in the fluid, step 1130, the controller can be activated such that the controller outputs an asymmetric biphasic signal, step 1140.

In one embodiment of the invention, a predetermined treatment program can be run, step 1150. Once the predetermined treatment program has been completed, at least one of the extremities can be removed from the fluid, step 1160. Once at least one extremity has been removed from the fluid, the extremity may be dried, optional step 1170. In some embodiments, the extremity does not have to be dried. After the extremity has dried, a topical cream may be applied to the treated extremity. In one embodiment, the topical cream may include, for example, camphor and menthol. These ingredients may produce a heating sensation followed by a cooling sensation to the skin. In one embodiment, the cream may include about 10% camphor and approximately 10% menthol. In another embodiment, the cream may include at least one of the following ingredients: baby oil, lanolin, and petroleum, in addition to the camphor and the menthol. One example of a topical cream that can be used in connection with this method of treating neuropathy is Rebuilder Medical, Inc.'s Cooling Cream.

While various embodiments of the invention have been described above, it should be understood that they have been presented by way of example only, and not limitation. Thus, the breadth and scope of the present invention should not be limited by any of the above-described exemplary embodiments, but should be defined only in accordance with the following claims and their equivalents.

For example, while the fluid was described in many embodiments as being water, the fluid may be any fluid that conducts an electrical current and does not harm the skin of the patient. Additionally, while particular electrolytes were disclosed herein, any electrolytes can be used in connection with the apparatus for treating neuropathy as described herein.

Additionally, while the present invention was described in terms of treating neuropathy, the apparatus and methods of the present invention can be used to treat, for example, arthritis, carpel tunnel syndrome, and multiple sclerosis, or nerve damages from, for example, automobile accidents.

1. An apparatus, comprising:
a controller, the controller being configured to output an asymmetrical biphasic signal;
a first container and a second container, the first container and the second container being configured to hold a fluid;
a first electrode configured to be in electrical contact with the fluid held by the first container and a second electrode configured to be in electrical contact with the fluid held by the second container, the first electrode and the second electrode being configured to be coupled to the controller, and being configured to receive the asymmetric biphasic signal output from the controller.

2. The apparatus of claim 1, wherein the asymmetrical biphasic signal is generated using a transformer.

3. The apparatus of claim 1, wherein the fluid is contained within a water-absorbing medium, the water absorbing medium being placed in at least the first container.

4. The apparatus of claim 1, wherein the fluid is a water-electrolyte solution.

5. The apparatus of claim 4, wherein the water-electrolyte solution includes at least one of potassium, calcium, benfotiamine, magnesium, and colloidal silver.

6. The apparatus of claim 1, wherein the controller is configured to selectively output the asymmetrical biphasic signal in at least two of a pulsed mode, a continuous wave mode, and a surged mode.

7. The apparatus of claim 1, wherein the first container and the second container are formed as part of a unitary structure.

8. The apparatus of claim 1, wherein the first electrode and the second electrode are electrically isolated from a power source.

9. The apparatus of claim 1, wherein the asymmetric biphasic signal is output at approximately 7.83 Hz.

10. The apparatus of claim 1, wherein a voltage associated with the asymmetric biphasic signal is adjusted based on the resistance between the first electrode and the second electrode.

11. A method comprising:
outputting an asymmetrical biphasic signal;
receiving the asymmetrical biphasic signal at a first electrode and a second electrode, the first electrode being configured to be in electrical contact with a fluid disposed in a first container, and the second electrode being configured to be in electrical contact with a fluid disposed within a second container.

12. The method of claim 11, said outputting further including selectively outputting an asymmetrical biphasic signal in one of a pulsed mode, a continuous wave mode, and a surged mode.

13. The method of claim 11, the fluid disposed in the first container and the fluid disposed in the second container being water, the method further comprising:
adding electrolytes to the water disposed in the first container and the water disposed in the second container.

14. The method of claim 11, wherein the fluid is substantially contained within a water-absorbing medium.

15. The method of claim 11, further comprising:
propagating the asymmetrical biphasic signal through a first extremity and a second extremity.

16. The method of claim 11, wherein the asymmetrical biphasic signal is output at approximately 7.83 Hz.

17. A method for treating neuropathy, comprising:
placing a first extremity in a first container, the first container including a fluid;
placing a second extremity in a second container, the second container including the fluid;
outputting an asymmetrical biphasic signal from a controller to the first extremity and the second extremity via the fluid in the first container and the fluid in the second container.

18. The method of treating neuropathy as recited in claim 17, further comprising:
removing the first extremity from the first container; and
applying a topical cream to the first extremity.

19. The method of treating neuropathy as recited in claim 18 wherein applying a topical cream includes applying a topical cream including camphor and menthol.

20. The method of treating neuropathy as recited in claim 17, wherein outputting the asymmetrical biphasic signal includes outputting the asymmetrical biphasic signal in one of a continuous wave mode, a pulsed mode, and a surged mode.

21. The method of treating neuropathy as recited in claim 17, further comprising:
adding electrolytes to the fluid in the first container.

22. The method of treating neuropathy as recited in claim 17, further comprising:
adding at least one of magnesium, calcium, potassium, and benfotiamine to the fluid in the first container.
